(19) United States
(12) Patent Application Publication
(10) Pub. No.: US 2014/0251342 A1
(43) Pub. Date: Sep. 11, 2014

(54) EYE PROTECTION DEVICE FOR PATIENTS UNDERGOING GENERAL ANESTHESIA IN DURING MEDICAL AND/OR SURGICAL PROCEDURES

(71) Applicant: Joseph O'Brien, North Oaks, MN (US)

(72) Inventor: Joseph O'Brien, North Oaks, MN (US)

(73) Assignee: SoJo Medical, North Oaks, MN (US)

(21) Appl. No.: 13/786,897

(22) Filed: Mar. 6, 2013

(51) Int. Cl. A61F 9/04; A61F 9/06

(52) U.S. Cl. CPC A61F 9/045; A61F 9/068
USPC 128/847

(57) ABSTRACT

An eye cover is provided that attaches to an air pump that provides a constant high humidity environment to the eyes, and protects the eyes especially during periods of anesthesia.

### BACKGROUND

1. Field of the Invention

This invention relates to a method and apparatus for eye protection. In particular, the invention relates to a method and apparatus to provide a protective environment for the eyes of patient undergoing a medical or surgical procedure, especially when the patient is under anesthesia. Of course, a person of ordinary skill in the art will understand that the invention is not necessarily so limited.

2. Background of the Invention

Eye injuries to patients who undergo general anesthesia are somewhat common. While a number of more serious eye injuries can result from general anesthesia, the most common injury is corneal abrasion. Although less serious than corneal abrasion, post surgical eye irritation is extremely common. In rare instances, an injury leading to loss of eyesight is possible. In these instances, pressure is accidentally placed on the eyeball during surgery, causing injury to the eye or blocking the eye's blood supply.

The cornea is the superficial clear outer layer of cells on the eyeball. A corneal abrasion is a scratch or tear in the cornea. The cornea is a sensitive area, and therefore corneal abrasions can cause mild to severe discomfort, irritation, and pain. It is not uncommon for a patient to describe the pain from the corneal abrasion as worse than the pain from the surgery. Additionally, corneal abrasion may also effect normal vision. While corneal abrasions often heal without long term effects on vision, severe abrasions may leave a scar on the cornea, which may be noticed as a dark or blurred spot in normal vision.

During general anesthesia, the eyelids of some patients do not fully close. This occurs in approximately 6 out of 10 patients. Additionally, under anesthesia the eyelids do not reflexively blink to keep the cornea moist. As a result, the cornea becomes very dry, compared to its normal condition. Various mechanisms have been suggested that may contribute to operative corneal abrasions. Analgesia and anesthesia necessarily mask the natural pain response, preventing the patient from sensing and reacting in a timely manner to the irritants and harmful stimulus thereby exacerbating such problems. Incomplete eyelid closure is a common phenomenon and may occur in as many as half of patients under general anesthesia, increasing corneal exposure and surface drying. The flow of oxygen or other gas from an anesthesia mask can flow over the eyes causing further drying and irritation to the eyes. Also, general anesthetics cause a significant decrease in tear production, which lubricates and protects the cornea. The cornea may be damaged by inadvertent pressure, or by chemicals such as the sterile prep. Damage can also result from such things as the oxygen facemask, laryngoscope, sterile drape, nasal cannula, low-hanging identification badges, or if the patient attempts to rub their eyes with a finger or a finger with a pulse-oximetered located thereon. The exact mechanism of injury in many cases is never actually known.

Other risk factors for corneal abrasions resulting from surgical procedures have been identified, and include, lateral or prone intraoperative patient positioning, the length of the surgery, head and neck procedures, and even the day of the week the surgery is performed (Monday being the worst day).

Attempts have been made in the prior art to address this problem; however, they suffer from a number of drawbacks. Medical professionals take care to avoid corneal abrasions during surgery. Gel is often applied to the eyes for lubrication, and the eyes are typically taped closed using gauze and tape. Some adhesive eye patch devices exist in the marketplace.

Despite these best known efforts, injuries and irritation are still common. Additionally, the use of devices such a gel and gauze pads, that might prevent mechanical trauma to the eye, often themselves cause post-surgical eye redness, irritation, and pain in up to 50% of surgical cases.

Studies have shown that serious corneal abrasions requiring treatment occur in as many as 1 in 1,750 surgical cases. In certain procedures, including brain and spinal surgery, this number has been described as 1 in 580 procedures. Under microscopic investigation, microscopic corneal abrasion in as many as 1 in 25 surgical procedures have been discovered.

Accordingly, a need exists for an improved method and apparatus to prevent corneal abrasion and trauma that result from surgery and medical treatment, especially when the patient is under anesthesia.

### SUMMARY OF THE INVENTION

An object of the present invention is to provide a method and apparatus to prevent corneal abrasion and trauma that result from surgery and medical treatment, especially when the patient is under anesthesia system that substantially eliminates the problems of the prior art.

These and other objects of the present invention will become apparent to those skilled in the art upon reference to the following specification, drawings, and claims. To that end, an eye cover is provided that attaches to an air pump that provides a constant high humidity environment to the eyes as well as providing protection from mechanical injury and undue pressure on the eyeball.

### BRIEF DESCRIPTION OF DRAWINGS

FIG. 1 shows an air pump and eye cover of the present invention.

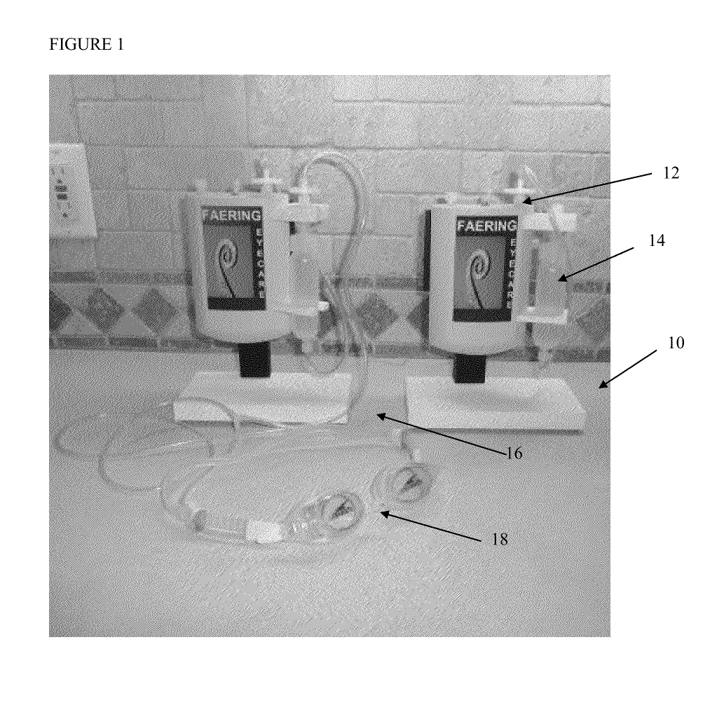

FIG. 2 shows the air pump, reservoir, and tubing.

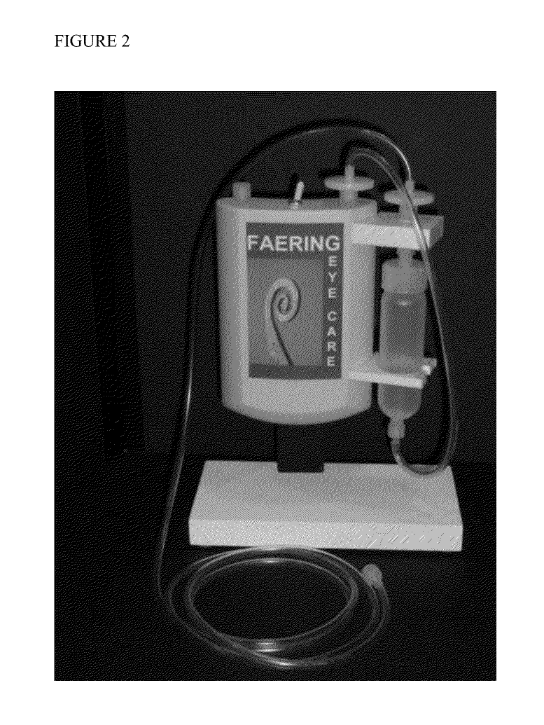

FIG. 3 shows the reservoir and tubing.

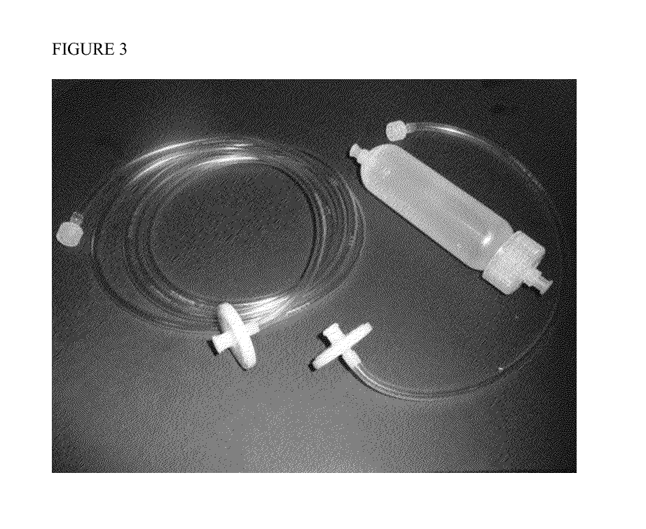

FIG. 4 shows the eye cover, adhesive sections, and tubing.

FIG. 5 shows the eye cover, adhesive sections, tubing on a mock patient.

FIG. 6 shows vents of the eye cover.

### DETAILED DESCRIPTION OF THE PREFERRED EMBODIMENTS

In the Figures, is shown a device 10 for the prevention of injury to the eye, and in particular to the eye(s) of a patient under anesthesia. The device 10 comprises a pump 12. The pump 12 is preferably an air pump, and more preferably a low velocity filtered air pump of a kind conventionally available.

Affixed to the pump 12 is an reservoir 14 connected with tubing 16 to the pump 12. The reservoir 14 is used to store a liquid that under pressure from the pump 12 provides highly humidified air preferably, or alternatively the liquid can processed by a nebulizer to provide a liquid mist. Tubing 16 extends, from the end of the reservoir 14 opposite the pump 12, to an eye cover 18. This connection allows the pump 12 to force high humidity solution from the reservoir 14 to the eye cover 18 to saturate the eyes of the patient. The eye cover 18 is worn by a patient during a medical or surgical procedure, and in particular one involving the use of anesthesia, thereby providing protection to the patient's eyes.

The eye cover 18 not only provides a physical barrier to protect the eyes from mechanical trauma and from pressure to the eyeball, but also forms a sufficient seal to contain the solution within the interior of the eye cover 18 to lubricate the eye so as to prevent damage due to dry eye conditions.

Thus, the invention consists of enclosed eye cover 18 or patches which cover the eyes of the patient and contact the face at the skin around the eye socket. The eye cover 18 can include a single cover over both eyes, or a separate cover over each eye (like swimmer's goggles). In either design, a volume of air-space is maintained between the open eye and the eye cover 18. The eye cover 18 could be made of silicone or other plastic material. Generally, flexible material is preferred; however, the eye cover 18 should preferably be strong and stiff enough to protect against trauma and pressure to the eyes. The eye cover 18 will be held in place using a head strap, or as explained in greater detail below adhesive can be used.

The eye cover 18 could be opaque, but more preferably transparent to allow the medical profession to examine the eyes through the eye cover 18.

A connector, such as a quick connect or medical leur, is attached to the side or nose bridge of the eye cover 18 allowing the connection of standard medical tubing 16. The connector communicates via a channel in the eye cover 18 through the side or nose bridge adjacent to the enclosed air-space in front of each eye.

The pump 12, which generates and pumps highly humidified air, is connected to the eye cover device via tubing 16. When activated, the pump 12 generates and pumps highly humidified air into the enclosed space in front of each eye.

The solution used with the present invention can comprise water, saline solution, or something like a tear replacement solution to best imitate natural tears. The solution should closely match the human tear to best protect the eye from dryness associated with corneal abrasions. In this regard, the human eye has a pH of approximately 7.5 and the pH of the solution should be substantially the same, and most preferably the solution would be substantially isotonic thereby avoiding any problems associated with hyper or hypo tonic solutions. Water has a pH of 7.0, which can be adequate in some circumstances. The solution should have a pH similar to the human eye in the preferred embodiment. The solution should preferably have an osmolarity similar to the human tear in the preferred embodiment, but may vary therefrom. Preferably, the humidity inside the eye cover 18 should be near 100% in the preferred embodiment, but may vary from a lower humidity to 100%.

The pumping velocity should be low in the preferred embodiment, so as not to dry the eye, but high enough to maintain a sufficiently high humidity level. The pump may be used continuously, or periodically as needed to achieve optimum saturation and humidity levels. Further, the eye cover 18 can be equipped with a pressure controlled vent to allow for some control of the pressure inside the eye cover 18. The invention can also include tubing to re-circulate the air/solution inside the eye cover 18 to the pump 12 (in which case a re-circulating pump can be used).

The air may be filtered in the preferred embodiment prior to or after the solution stage, or not. The humidified air may contain droplets of liquid in the preferred embodiment, but may not contain droplets of liquid. A mendicant, such as steroid, antihistamine, anesthetic, antibiotic, etc can be used as well. The solution most preferably should be sterile in the preferred embodiment, but that may not necessarily be the case.

As stated above, in an additional embodiment of the invention the device 10 could introduce a medication into the humidified vapor in order to sooth the eye, or treat a condition. In a further embodiment of the device 10 a sensor can sense the relative humidity of the enclosed air space and automatically adjust to a pre-defined optimal level, but turning the pump on or off, by altering the velocity of the pump, or by opening a vent on the eye cover 18, or in any other manner. In yet another embodiment, the device 10 could sense temperature and heat or cool the humidified air to maintain an optimal temperature.

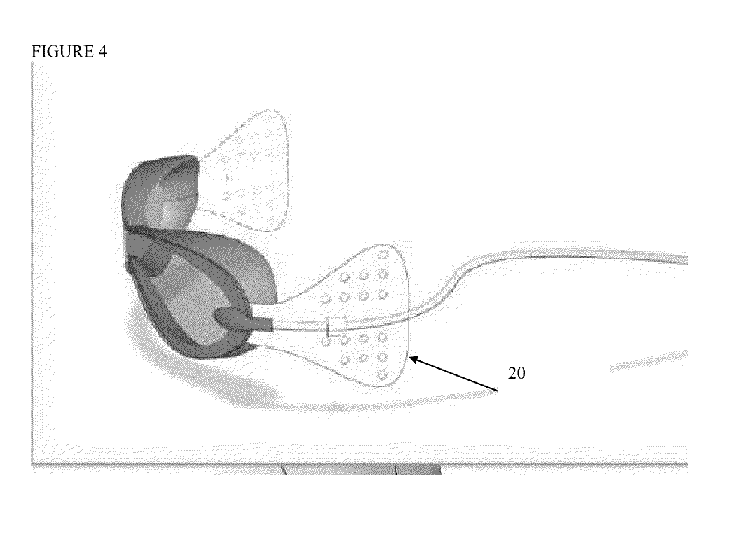

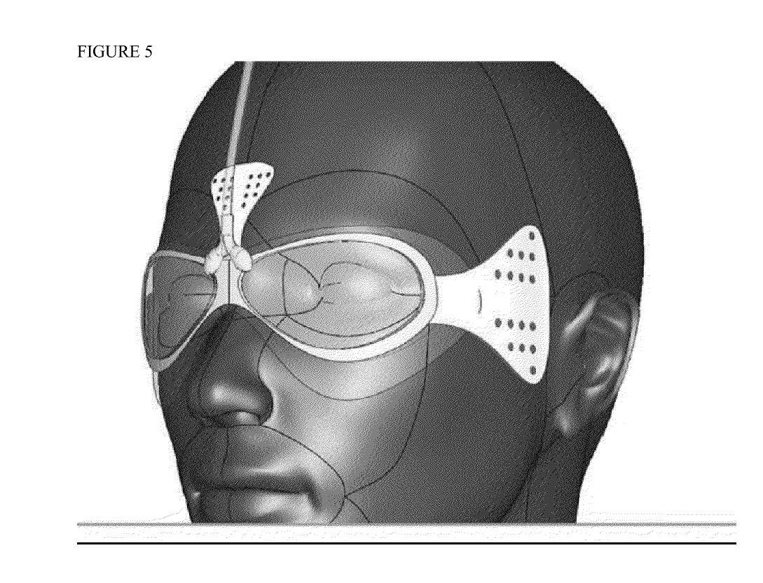

Additional embodiments of the present invention are show in FIGS. 4-6. In particular, the eye cover 18 includes adhesive sections 20 to retain the eye cover 18 on the patient. The adhesive sections 20 are comprised of a conventional adhesive sufficient to removably secure the eye cover 18. The adhesive sections 20 can be on the sides of the eye cover 18, or in the middle, or some combination thereof. The adhesive sections 20 can form a seal sufficient to retain the humidified solution within the interior of the eye cover 18.

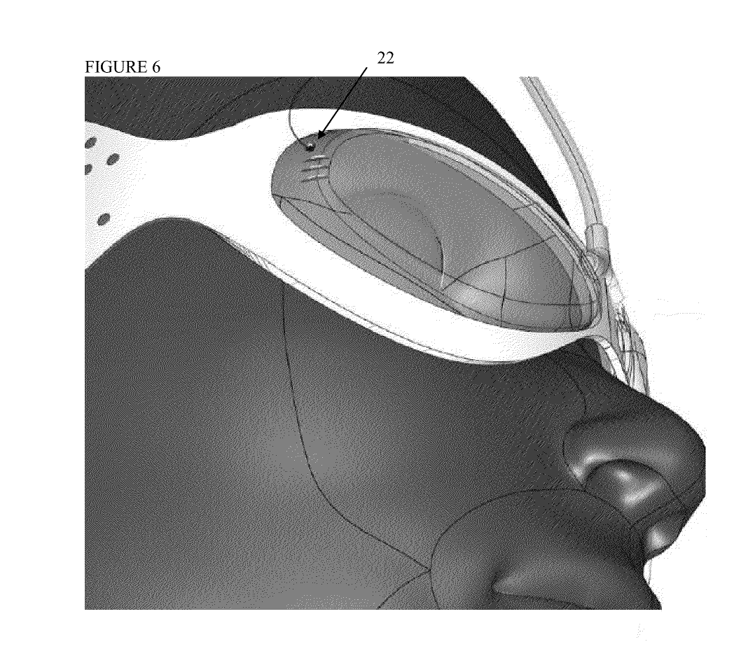

Tubing 16 can attach to the sides or middle of the eye cover 18, as shown in the Figures. Additionally, as shown in FIG. 6 the eye cover 18 can include a vent 22, which will allow the humidified air inside the eye cover 18 to escape are some predetermined pressure. This will provide greater flexibility in using the pump 12. For example, the pump 12 can be used continuously, and provide a refreshed humidified airflow. Furthermore, the eye cover 18 shown in FIG. 6 includes separate chambers for each eye and the tubing 16 splits to provide high humidity air to both chambers.

The use of adhesive sections 20 is particular advantageous given the nature of the use of the invention. For use with a person in a prone position of a person under anesthesia the eye cover 18 with adhesive sections 20 would be easier to apply and remove than a strap, and is less likely to slip and/or need repositioning (if, for example the patient need to be moved while under anesthesia). A strap can work with the present invention in many situations.

The advantages of this present invention are that it allows the eyes to be mechanically protected and kept moist without any contact with the cornea or pressure to the eyeball of the patient. It is anticipated that the invention will prevent most or all corneal abrasions related to anesthesia in surgery, as well as protect the eyes from the rare and more serious pressure related injuries.

Unless otherwise defined, all technical and scientific terms used herein have the same meaning as commonly understood by one of ordinary skill in the art to which this invention belongs. Although methods and materials similar to or equivalent to those described herein can be used in the practice or testing of the present invention, suitable methods, and materials are described below. All publications, patent applications, patents, and other references mentioned herein are incorporated by reference in their entirety to the extent allowed by applicable law and regulations. In case of conflict, the present specification, including definitions, will control.

The present invention may be embodied in other specific forms without departing from the spirit or essential attributes thereof, and it is therefore desired that the present embodiment be considered in all respects as illustrative and not restrictive, reference being made to the appended claims rather than to the foregoing description to indicate the scope of the invention. Those of ordinary skill in the art that have the disclosure before them will be able to make modifications and variations therein without departing from the scope of the invention.

1. A device for protecting the eyes of a person under anesthesia, comprising
a pump for supplying forced air;
an reservoir containing a liquid solution and in operative communication with said pump;
an eye cover for protecting the eyes of a person in operative communication with reservoir to provide a high humidity environment during a period of anesthesia.

2. The device of claim 1 where flexible tubing provides operatively communicating between the reservoir and the eye cover.

3. The device of claim 1 further comprising a filter to filter the air from the pump.

4. The device of claim 1 wherein the eye cover covers both eyes.

5. The device of claim 1 wherein the eye cover has separate chambers for each eye.

6. The device of claim 1 wherein the eye cover further comprises a vent.

7. The device of claim 1 further comprising a humidity sensor to sense the humidity level of the eye cover and adjusting the level by controlling the pump.

8. The device of claim 1 further comprising a temperature sensor to sense the temperature of the solution.

9. The device of claim 1 further comprising a strap affixed to the eye cover to hold the eye cover in place.

10. The device of claim 1 further comprising an adhesive section affixed to the eye cover to hold the eye cover in place.

11. The device of claim 10 further comprising multiple adhesive sections.

12. The device of claim 1 wherein the high humidity environment is comprised of humid air.

13. The device of claim 1 further providing a nebulizer and the high humidity environment is comprised of a mist.

14. The device of claim 1 wherein protecting the eyes includes protection from applied pressure.

15. A method of for protecting the eyes of a person under anesthesia, comprising
securing an eye cover to a patient; and
pumping a high humidity solution to the eye cover from a pump.

16. The method of claim 15 providing an reservoir for containing the solution.

17. The method of claim 16 providing tubing for connecting the pump, reservoir, and eye cover.

18. The method of claim 15 wherein the eye cover comprises an adhesive section affixed to the eye cover to hold the eye cover in place.
